(19) United States
(12) Patent Application Publication
(10) Pub. No.: US 2021/0041429 A1
(43) Pub. Date: Feb. 11, 2021

(54) TRANSPORTATION AND DETECTION OF ANALYTES

(71) Applicant: AMMR Joint Venture, La Jolla, CA (US)

(72) Inventors: Regis Paytavi, Costa Mesa, CA (US); Kiana Aran, Pasadena, CA (US); Brett Goldsmith, San Diego, CA (US); Alexander Kane, Santa Cruz, CA (US)

(21) Appl. No.: 16/987,997

(22) Filed: Aug. 7, 2020

Related U.S. Application Data
(60) Provisional application No. 62/883,887, filed on Aug. 7, 2019.
(60) Provisional application No. 63/036,772, filed on Jun. 9, 2020.

(51) Int. Cl. G01N 33/543; G01N 27/74

(52) U.S. Cl. CPC G01N 33/54333; G01N 33/54366; G01N 27/745

(57) ABSTRACT

Apparatuses, systems, and methods are disclosed for transportation and detection of analytes. Beads may be functionalized with a capture moiety to bind to a target moiety. Beads that have not been incubated in a sample solution may be positioned in a fluid, near a sensing surface for a biosensor. A calibration measurement may be performed using the biosensor, after which the beads may be removed. Beads that have been incubated in the sample solution may be positioned near the sensing surface, and a detection measurement may be performed using the biosensor. A parameter such as the presence, absence or concentration of the target moiety in the sample solution may be determined based on the calibration measurement and the detection measurement.

### CROSS-REFERENCES TO RELATED APPLICATIONS

This application claims the benefit of U.S. Provisional Patent Application No. 62/883,887 entitled “DEVICES AND METHODS FOR LABEL-FREE DETECTION OF ANALYTES” and filed on Aug. 7, 2019 for Regis Peytavi et al.; and claims the benefit of U.S. Provisional Patent Application No. 63/036,772 entitled “DYNAMIC EXCITATION AND MEASUREMENT OF BIOCHEMICAL INTERACTIONS” and filed on Jun. 9, 2020 for Kiana Aran et al.; each of which is incorporated herein by reference in their entireties to the extent legally allowable.

### FIELD

The subject matter disclosed herein relates to biotechnology and more particularly relates to transportation and detection of analytes.

### BACKGROUND

Various biochemical assays exist for detecting analytes, such as certain molecules or moieties. Certain assays may detect analytes in a liquid solution when the analytes are near a sensing surface. However, many analytes in the liquid solution may not be sufficiently close to the sensing surface to be detected.

### SUMMARY

Systems are disclosed for transportation and detection of analytes. In some embodiments, a chip-based field effect biosensor includes a sensing surface. In some embodiments, a sensing surface is configured so that one or more output signals for the chip-based field effect biosensor are affected by electrical charges within a measurement distance of the sensing surface, in response to application of one or more excitation conditions to the chip-based field effect biosensor and application of a fluid in contact with the sensing surface. In some embodiments, a bead control device includes one or more bead control components for electromagnetically positioning a plurality of beads within the fluid. In some embodiments, the beads may be functionalized with a capture moiety to bind to a target moiety. In some embodiments, a measurement controller is configured to operate the chip-based field effect biosensor and the bead control device to perform a calibration measurement of at least one of the output signals with a first set of the beads positioned within the measurement distance of the sensing surface, where the first set of the beads has not been incubated in a sample solution. In some embodiments, the measurement controller is configured to operate the bead control device to remove the first set of the beads from the sensing surface. In some embodiments, the measurement controller is configured to operate the chip-based field effect biosensor and the bead control device to perform a detection measurement of the at least one output signal with a second set of the beads positioned within the measurement distance of the sensing surface, where the second set of the beads has been incubated in the sample solution. In some embodiments, an analysis module is configured to determine a parameter relating to presence of the target moiety in the sample solution, based on the calibration measurement and the detection measurement.

Methods are disclosed for transportation and detection of analytes. In some embodiments, a method includes providing a plurality of beads functionalized with a capture moiety to bind to a target moiety. In some embodiments, a method includes positioning a first set of the beads within a fluid to be within a measurement distance of a sensing surface of a chip-based field effect biosensor, where the first set of the beads has not been incubated in a sample solution. In some embodiments, a method includes performing a calibration measurement of at least one output signal from the chip-based field effect biosensor. In some embodiments, a method includes removing the first set of the beads from the sensing surface. In some embodiments, a method includes incubating a second set of the beads in the sample solution. In some embodiments, a method includes positioning the second set of the beads within the fluid to be within the measurement distance of the sensing surface. In some embodiments, a method includes performing a detection measurement of the at least one output signal. In some embodiments, a method includes determining a parameter relating to presence of the target moiety in the sample solution, based on the calibration measurement and the detection measurement.

Apparatuses are disclosed for transportation and detection of analytes. In some embodiments, an apparatus includes means for positioning a plurality of beads, within a fluid, within a measurement distance of a sensing surface of a chip-based field effect biosensor, where the beads are functionalized with a capture moiety to bind to a target moiety. In some embodiments, an apparatus includes means for performing a calibration measurement using the chip-based field effect biosensor, with a first set of the beads positioned within the measurement distance of the sensing surface, where the first set of the beads has not been incubated in a sample solution. In some embodiments, an apparatus includes means for performing a detection measurement using the chip-based field effect biosensor, with a second set of the beads positioned within the measurement distance of the sensing surface, where the second set of the beads has been incubated in the sample solution.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a schematic block diagram illustrating one embodiment of a system for transportation and detection of analytes;

FIG. 2 is a schematic block diagram illustrating one embodiment of an apparatus for transportation and detection of analytes, including one embodiment of a biologically gated transistor;

FIG. 3 is a schematic block diagram illustrating another embodiment of an apparatus for transportation and detection of analytes, including another embodiment of a biologically gated transistor;

FIG. 4 is a schematic block diagram illustrating a further embodiment of an apparatus for transportation and detection of analytes, including embodiments of beads and bead control components;

FIG. 5 is a schematic block diagram illustrating another embodiment of an apparatus for transportation and detection of analytes, including embodiments of beads and bead control components;

FIG. 6 is a side view illustrating one embodiment of beads;

FIG. 7 is a detail view of a region indicated in FIG. 4, illustrating beads and a sensing surface during a calibration measurement, in one embodiment;

FIG. 8 is a detail view of a region indicated in FIG. 4, illustrating removal of beads from a sensing surface, in one embodiment;

FIG. 9 is a detail view of a region indicated in FIG. 4, illustrating beads and a sensing surface during incubation, in one embodiment;

FIG. 10 is a detail view of a region indicated in FIG. 4, illustrating beads and a sensing surface during a detection measurement, in one embodiment;

FIG. 11 is a schematic block diagram illustrating one embodiment of an apparatus including a bead control device and a measurement controller;

FIG. 12 is a schematic flow chart diagram illustrating one embodiment of a method for transportation and detection of analytes;

FIG. 13 is a schematic flow chart diagram illustrating another embodiment of a method for transportation and detection of analytes; and

FIG. 14 is a schematic flow chart diagram illustrating another embodiment of a method for transportation and detection of analytes.

### DETAILED DESCRIPTION

As will be appreciated by one skilled in the art, aspects of the disclosure may be embodied as a system, method, or program product. Accordingly, embodiments may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, embodiments may take the form of a program product embodied in one or more computer readable storage devices storing machine readable code, computer readable code, and/or program code, referred hereafter as code. The storage devices may be tangible, non-transitory, and/or non-transmission. The storage devices may not embody signals. In a certain embodiment, the storage devices only employ signals for accessing code.

Certain of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in code and/or software for execution by various types of processors. An identified module of code may, for instance, comprise one or more physical or logical blocks of executable code which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different computer readable storage devices. Where a module or portions of a module are implemented in software, the software portions are stored on one or more computer readable storage devices.

Any combination of one or more computer readable medium may be utilized. The computer readable medium may be a computer readable storage medium. The computer readable storage medium may be a storage device storing the code. The storage device may be, for example, but not limited to, an electronic, magnetic, optical, electromagnetic, infrared, holographic, micromechanical, or semiconductor system, apparatus, or device, or any suitable combination of the foregoing.

More specific examples (a non-exhaustive list) of the storage device would include the following: an electrical connection having one or more wires, a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Code for carrying out operations for embodiments may be written in any combination of one or more programming languages including an object oriented programming language such as Python, Ruby, Java, Smalltalk, C++, or the like, and conventional procedural programming languages, such as the “C” programming language, or the like, and/or machine languages such as assembly languages. The code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A component, as used herein, comprises a tangible, physical, non-transitory device. For example, a component may be implemented as a hardware logic circuit comprising custom VLSI circuits, gate arrays, or other integrated circuits; off-the-shelf semiconductors such as logic chips, transistors, or other discrete devices; and/or other mechanical or electrical devices. A component may also be implemented in programmable hardware devices such as field programmable gate arrays, programmable array logic, programmable logic devices, or the like. A component may comprise one or more silicon integrated circuit devices (e.g., chips, die, die planes, packages) or other discrete electrical devices, in electrical communication with one or more other components through electrical lines of a printed circuit board (PCB) or the like. Each of the modules described herein, in certain embodiments, may alternatively be embodied by or implemented as a component.

A circuit, or circuitry, as used herein, comprises a set of one or more electrical and/or electronic components providing one or more pathways for electrical current. In certain embodiments, circuitry may include a return pathway for electrical current, so that a circuit is a closed loop. In another embodiment, however, a set of components that does not include a return pathway for electrical current may be referred to as a circuit or as circuitry (e.g., an open loop). For example, an integrated circuit may be referred to as a circuit or as circuitry regardless of whether the integrated circuit is coupled to ground (as a return pathway for electrical current) or not. In various embodiments, circuitry may include an integrated circuit, a portion of an integrated circuit, a set of integrated circuits, a set of non-integrated electrical and/or electrical components with or without integrated circuit devices, or the like. In one embodiment, a circuit may include custom VLSI circuits, gate arrays, logic circuits, or other integrated circuits; off-the-shelf semiconductors such as logic chips, transistors, or other discrete devices; and/or other mechanical or electrical devices. A circuit may also be implemented as a synthesized circuit in a programmable hardware device such as field programmable gate array, programmable array logic, programmable logic device, or the like (e.g., as firmware, a netlist, or the like). A circuit may comprise one or more silicon integrated circuit devices (e.g., chips, die, die planes, packages) or other discrete electrical devices, in electrical communication with one or more other components through electrical lines of a printed circuit board (PCB) or the like. Each of the modules described herein, in certain embodiments, may be embodied by or implemented as a circuit.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to,” unless expressly specified otherwise. An enumerated listing of items does not imply that any or all of the items are mutually exclusive, unless expressly specified otherwise. The terms “a,” “an,” and “the” also refer to “one or more” unless expressly specified otherwise.

Furthermore, the described features, structures, or characteristics of the embodiments may be combined in any suitable manner. In the following description, numerous specific details are provided, such as examples of programming, software modules, user selections, network transactions, database queries, database structures, hardware modules, hardware circuits, hardware chips, etc., to provide a thorough understanding of embodiments. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an embodiment.

Aspects of the embodiments are described below with reference to schematic flowchart diagrams and/or schematic block diagrams of methods, apparatuses, systems, and program products according to embodiments. It will be understood that each block of the schematic flowchart diagrams and/or schematic block diagrams, and combinations of blocks in the schematic flowchart diagrams and/or schematic block diagrams, can be implemented by code. This code may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the schematic flowchart diagrams and/or schematic block diagrams block or blocks.

The code may also be stored in a storage device that can direct a computer, other programmable data processing apparatus, or other devices to function in a particular manner, such that the instructions stored in the storage device produce an article of manufacture including instructions which implement the function/act specified in the schematic flowchart diagrams and/or schematic block diagrams block or blocks.

The code may also be loaded onto a computer, other programmable data processing apparatus, or other devices to cause a series of operational steps to be performed on the computer, other programmable apparatus or other devices to produce a computer implemented process such that the code which execute on the computer or other programmable apparatus provide processes for implementing the functions/acts specified in the flowchart and/or block diagram block or blocks.

The schematic flowchart diagrams and/or schematic block diagrams in the Figures illustrate the architecture, functionality, and operation of possible implementations of apparatuses, systems, methods, and program products according to various embodiments. In this regard, each block in the schematic flowchart diagrams and/or schematic block diagrams may represent a module, segment, or portion of code, which comprises one or more executable instructions of the code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and code.

The description of elements in each figure may refer to elements of proceeding figures. Like numbers refer to like elements in all figures, including alternate embodiments of like elements.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C.” As used herein, “a member selected from the group consisting of A, B, and C and combinations thereof” includes only A, only B, only C, a combination of A and B, a combination of B and C, a combination of A and C or a combination of A, B and C.

### Definitions

The term “chip-based field effect biosensor,” as used herein, refers to a sensor that includes a sensing surface on a substrate, such that when a fluid is applied in contact with the sensing surface, an output signal for the biosensor is capable of being modulated or affected by electric and/or magnetic fields in a fluid, proximate to the sensing surface. For example, ions or polar molecules within the fluid may affect the electric field near the sensing surface, thus affecting an output signal such as a voltage, current, impedance, capacitance, or the like. The term “biosensor” may refer to such a device in use, with a fluid applied to the sensing surface, or to the same device before the fluid has been applied. The term “biosensor” may be used without regard to whether molecules or moieties within the fluid are biologically produced. For example, a biosensor may be used to sense biologically produced or synthetically produced molecules or moieties in the fluid, but may in either case still be referred to as a “biosensor.”

The term “biologically gated transistor,” as used herein, refers to a type of chip-based field effect biosensor, configured as a transistor where current between source and drain terminals, through at least one channel, is capable of being gated, modulated, or affected by events, occurrences, or interactions within a fluid in contact with a surface of the channel. Thus, a channel surface is a sensing surface for the biosensor. For example, an interaction of ions, molecules, or moieties within the fluid, or an interaction between the channel surface and ions, molecules, or moieties within the fluid, may be capable of gating, modulating, or effecting the channel current. The term “biologically gated transistor” may be used to refer to such a device in use, with a fluid applied to the surface of the channel, or to the same device before the fluid has been applied. The term “biologically gated transistor” may be used without regard to whether molecules or moieties within the fluid are biologically produced. For example, a biologically gated transistor may be gated by interactions between a biologically produced enzyme in the fluid and the enzyme's substrate, or may be gated by non-biological interactions within the fluid, but may still be referred to as “biologically gated.”

The term “output signal,” as used herein, refers to a measurable or detectable electrical signal from a chip-based field effect biosensor, or to a result that can be calculated based on the measurable or detectable signal. For example, an output signal may be a voltage at one or more terminals of a chip-based field effect biosensor, a current at one or more chip-based field effect biosensor, a capacitance, inductance, or resistance (calculated based on applied and measured voltages and currents), a complex-valued impedance, a complex impedance spectrum, an electrochemical impedance spectrum, a threshold voltage, a Dirac voltage, a power spectral density, one or more network parameters (such as S-parameters or h-parameters), or the like.

The term “distance,” as used herein with reference to a distance from a surface such as a sensing surface in a chip-based field effect biosensor or the surface of a channel in a biologically gated transistor, refers to a distance between a point (e.g., in the fluid applied to a biosensor), and the closest point of the surface to that point. For example, the distance from a sensing surface to a point directly above the sensing surface in the applied fluid is the distance between a point on the sensing surface to the point in the fluid, along a line that is normal (perpendicular) to the sensing surface.

The term “measurement distance,” as used herein, refers to a distance from the sensing surface in a chip-based field effect biosensor, such that at least some interaction, molecule or moiety occurring at or within the measurement distance affects an output signal in a way that is detectable by a measurement controller. In other words, output signals from a chip-based field effect biosensor are sensitive to charges (e.g., of ions or within moieties, molecules, or complexes of molecules) within the measurement distance. Whether an effect on an output signal is detectable by a measurement controller may depend on actual sensitivity of the measurement controller, on a noise level for noise in the output signal, the extent to which the output signal is affected by events or occurrences closer to the sensing surface, or the like. Whether an effect on an output signal is detectable by a measurement controller may be based on a predetermined threshold for detection or sensitivity, which may be signal to noise ratio, a ratio between effects on the output signal caused by events at a distance from the surface to effects on the output signal caused by events at the sensing surface, or the like. In some examples, a measurement distance may depend on excitation conditions, or may be frequency dependent.

The term “within the measurement distance,” as used herein, refers to objects within a fluid applied to chip-based field effect biosensor, such that a distance from the sensing surface to at least a portion of such an object is less than the measurement distance. For example, a bead in the fluid may be referred to as being within the measurement distance, if at least a part of the bead is closer than the measurement distance to the surface. Such a bead may be entirely within the measurement distance, or may include a portion that extends further away from the sensing surface than the measurement distance.

The term “excitation condition,” as used herein, refers to a physical, electrical, or chemical condition applied to a chip-based field effect biosensor or to a sample for measurement by a chip-based field effect biosensor. Excitation conditions may affect a state of a molecules or moieties in the fluid applied to the biosensor, which in turn may affect one or more output signals from the biosensor. For example, excitation conditions may include voltages, currents, frequencies, amplitudes, phases, or waveforms of electrical signals applied to a biologically gated transistor, one or more temperatures, one or more fluid flow rates, one or more wavelengths of electromagnetic radiation, or the like.

The term “beads,” as used herein, refers to particles in the range of about 1 nm to 10 μm in diameter having a functionalized surface configured to bind with a corresponding component of a molecule or moiety in solution. Some beads are magnetic and other beads are non-magnetic. Non-limiting examples of beads include particles functionalized with a streptavidin coating configured to bind with biotinylated molecules in solution. Other non-limiting examples of materials for functionalizing a bead surface include antibodies, biotin, proteins that bind to biotin, zinc finger proteins, CRISPR Cas family enzymes, nucleic acids, and synthetic nucleic acid analogs such as peptide nucleic acid, xeno nucleic acid, and the like.

The term “moiety,” as used herein, refers to a part of a molecule. For example, a moiety may be a biotin portion of a biotinylated molecule, a streptavidin moiety linked to a surface of a bead, or the like. In the plural form, the term “moieties” may be used to refer to multiple types of moiety (e.g., a capture moiety and a target moiety) or to multiple instances of the same type of moiety for multiple molecules (e.g., multiple instances of the a target moiety).

The term “target moiety,” as used herein refers to a moiety of an analyte, which may be a molecule or molecular complex for which the presence, absence, concentration, activity, or other parameters relating to the analyte may be determined in an assay or test. For example, an assay using a chip-based field effect biosensor may be used to determine the presence, absence, or concentration of an analyte that includes the target moiety.

The term “capture moiety,” as used herein, refers to a moiety with an affinity for binding to a target moiety. For example, the capture moiety may be a biotin-binding protein when the target moiety is biotin, or may be an RNA-guided Cas enzyme when the target moiety is a nucleic acid sequence. Conversely, the capture moiety may be biotin when the target moiety is a biotin-binding protein, or may be a nucleic acid sequence when the target moiety is an RNA-guided Cas enzyme.

Various biochemical assays exist for detecting analytes, such as certain molecules or moieties. Certain assays may detect analytes in a liquid solution when the analytes are near a sensing surface. However, when analytes are large molecules, diffusion of the analytes in the liquid solution may not bring enough of the analytes close enough to the sensing surface to be detected.

Additionally, some assays may involve functionalization of the sensing surface to capture or bind to the analytes. However, a sensing surface, once functionalized to bind to a particular analyte, may be unsuited for measurement of other analytes, with the result that manufacturers may make expensive single-purpose sensors rather than low-cost sensors capable of being used for multiple assays. Also where a functionalized sensing surface or an analyte is labeled with a fluorescent or colorimetric label to optically detect the binding of the analyte to the sensing surface, reagents for labeling, time for labeling reactions, and optical components for detection may add significantly to the time, complexity and expense of an assay.

By contrast, assays using chip-based field effect biosensors, as disclosed herein, with beads to capture target moieties and bead control components to position the beads near the sensing surface, may efficiently and inexpensively transport and detect analytes. Chip-based field effect biosensors may be built using traditional electronics manufacturing techniques, leading to lower costs. Systems using chip-based field effect biosensors may be capable of performing electronic target detection for a wide variety of targets, leading to lower overall cost for individual assays.

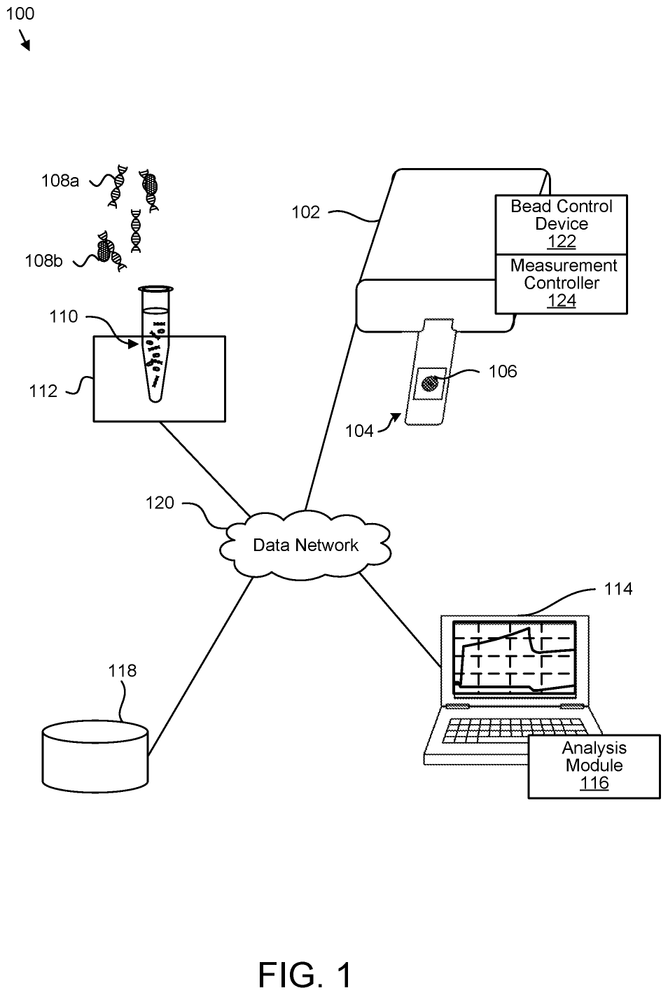

FIG. 1 is a schematic block diagram illustrating one embodiment of a system 100 for transportation and detection of analytes. The system 100, in the depicted embodiment, includes one or more chip-based field effect biosensors 104, a chip reader device 102, a sample prep apparatus 112, a computing device 114, a remote data repository 118, and a data network 120.

In the depicted embodiment, a chip-based field effect biosensor 104, in the depicted embodiment, includes one or more biologically gated transistors 106, which are described in further detail below. In various embodiments, a chip-based field effect biosensor 104 may include one or more sensing surfaces, arranged on a solid support. In a biologically gated transistor 106, a sensing surface may be a surface of a channel that couples a drain terminal to a source terminal. In a capacitive or electrochemical sensor, a sensing surface may be a surface of a working electrode, and the chip-based field effect biosensor 104 may include an electrochemical system with a reference electrode to measure an electrochemical potential and a counter electrode to modify an electrochemical potential.

One or more layers of ions may form near the sensing surface when a fluid is applied in contact with the sensing surface. For example, a double layer of ions may include a first layer of ions attracted or adsorbed to the sensing surface and a second layer of ions attracted to the ions in the first layer. Or, if the surface has been functionalized by immobilizing certain molecules or moieties (e.g., proteins, peptides, surfactants, polymers such as polyethylene glycol, or the like) to the sensing surface, forming an ion-permeable layer with a net charge, then ions from the fluid may diffuse into the ion-permeable layer of immobilized molecules or moieties due to the Gibbs-Donnan effect, forming a Donnan equilibrium region. In either case, charges near the sensing surface may act as a dielectric between the channel of a biologically gated transistor 106, or the working electrode of a capacitive sensor, and the bulk of the applied fluid.

When excitation conditions are applied to a chip-based field effect biosensor 104, output signals such as a channel current or capacitance may depend on charges within this (effective) dielectric layer, or more generally within a measurement distance of the sensing surface. Charges within a measurement distance of the sensing surface, which affect the output signals of the biosensor 104, may be positively or negatively charged ions or moieties, or may be neutrally charged molecules or moieties (e.g., including an equal number of positive and negative charges) that displace other charges. For example, if the fluid applied to the chip-based field effect biosensor 104 includes DNA molecules with negatively charged phosphate groups, then transporting the DNA molecules to be near or in contact with the sensing surface brings negative charges within the measurement distance, thus affecting the output signal(s) for the biosensor 104.

In some embodiments, a chip-based field effect biosensor 104 may include a plurality of transistors where at least one of the transistors is a biologically gated transistor 106. In some embodiments, a chip-based field effect biosensor 104 may include one or more additional sensors that do not use field-effect sensing, alongside sensors with sensing surfaces for field-effect sensing. For example, various types of sensors may be included that use terahertz spectroscopy, surface-enhanced spectroscopy, quartz crystal microbalance, grating-coupled interferometry, and so forth. In some embodiments, a chip-based field effect biosensor 104 may include further components such as a flow cell or fluid propulsion mechanism.

In the depicted embodiment, the chip reader device 102 includes circuitry for communicating with (e.g., sending electrical signals to or receiving electrical signals from) components of the chip-based field effect biosensor 104. For example, a chip-based field effect biosensor 104 may include a chip or integrated circuit with one or more biologically gated transistors 106, mounted to a printed circuit board with electrical contacts at one edge. A socket in the chip reader device 102 may include matching contacts, so that the chip-based field effect biosensor 104 can be plugged into or removed from the chip reader device 102. Various other or further types of connectors may be used to provide a detachable coupling between a chip-based field effect biosensor 104 and a chip reader device 102.

In a further embodiment, the chip reader device 102 may include circuitry for communicating via the data network 120. For example, the chip reader device 102 may communicate information about measurements performed using the chip-based field effect biosensor 104 to the computing device 114 and/or to a remote data repository 118, over the data network. The data network 120, in various embodiments, may be the Internet, or may be another network such as a wide area network, metropolitan area network, local area network, virtual private network, or the like. In another embodiment, the chip reader device 102 may communicate information in another way, in addition to or in place of communicating over a data network 120. For example, the chip reader device 102 may display or print information, save information to a removable data storage device, or the like.

In the depicted embodiment, a bead control device 122 and a measurement controller 124 are implemented by the chip-based field effect biosensor 104 and/or the chip reader device 102.

A bead control device 122, in various embodiments, may include one or more bead control components for electromagnetically positioning a plurality of beads, within a fluid applied to a chip-based field effect biosensor 104. Beads may be functionalized with a capture moiety to bind to a target moiety, as discussed in further detail with reference to subsequent figures, and may be controlled to bring the beads within the measurement distance of a sensing surface chip-based field effect biosensor 104. Thus, in various embodiments, beads may bind to an analyte, and may be electromagnetically positioned to bring the analyte close to the sensing surface to be detected.
Electromagnetically positioning beads, in various embodiments, may include using electric and/or magnetic fields to move beads, or to limit or constrain the motion of beads. For example, bead control components that electromagnetically position beads may be electromagnets that can be controlled to move magnetic beads toward or away form a surface, or to hold magnetic beads onto a surface (e.g., during fluid flow to wash the beads). As another example, bead control components that electromagnetically position beads may be a pair of parallel conductive plates (or other conductors) configured so that applying a different voltage to each of the conductors produces an electric field between the conductors, to move electrically charged beads or to limit the motion of the beads by attracting or repelling them. Various other or further components for producing electric and/or magnetic fields may be used as bead control components.

Additionally, in various embodiments, a bead control device 122 may include circuitry for controlling bead control components. For example, a bead control device 122 may include power supply components, current sources or regulators for controlling electromagnets, voltage sources or regulators for applying an electric potential to field plates, control circuitry for applying, removing, or modulating power to the bead control components, or the like.

A measurement controller 124, in various embodiments, may include excitation circuitry to apply excitation conditions to a chip-based field effect biosensor 104, including a biologically gated transistor 106 or a capacitive sensor. Output signals from the chip-based field effect biosensor 104 (such as electrical currents, voltages, capacitances, impedances, or the like) may be affected by charges within the measurement distance of a sensing surface, in response to the excitation conditions and the application of a fluid in contact with the sensing surface. For example, if the applied fluid contains biotinylated DNA, and if beads with a capture moiety that binds to the target biotin moiety are incubated in the fluid and brought within the measurement distance, then the negative charge of the DNA bound to the beads may affect one or more of the output signals. The measurement controller 124 may include measurement circuitry to perform one or more measurements of at least one of the output signals that are affected by the charges within the measurement distance. Various embodiments of a measurement controller 124 are described in further detail below.

In some embodiments, a chip-based field effect biosensor 104 may include the bead control device 122 and/or the measurement controller 124. For example, bead control components, excitation circuitry and/or measurement circuitry may be provided on the same chip as a biologically gated transistor 106 or a capacitive sensor, or on the same package, on the same printed circuit board, or the like, as part of a chip-based field effect biosensor 104. In another embodiment, the chip reader device 102 may include the bead control device 122 and/or the measurement controller 124. For example, bead control components, excitation circuitry and/or measurement circuitry may be provided in a chip reader device 102 so as to be reusable with multiple chip-based field effect biosensors 104.

In another embodiment, a chip-based field effect biosensor 104 and a chip reader device 102 may both include portions of the bead control device 122 and/or the measurement controller 124. For example, the chip-based field effect biosensor 104 may include portions of the bead control device 122 such as an electromagnet proximate to the sensing surface for positioning beads within the measurement distance of the sensing surface, and the and the chip reader device 102 may include other portions of the bead control device 122 such as an electromagnet for removing beads from the sensing surface. In various embodiments, portions of the bead control device 122 and/or the measurement controller 124 may be disposed between a chip-based field effect biosensor 104 and a chip reader device 102 in various other or further ways.

Additionally, although the system 100 in the depicted embodiment includes a chip-based field effect biosensor 104 that may be coupled to or removed from a chip reader device 102, the functions and/or components of a chip-based field effect biosensor 104 and a chip reader device 102 may be integrated into a single device in another embodiment. Conversely, in some embodiments, a system may include multiple devices rather than a single chip reader device 102. For example, excitation circuitry and/or measurement circuitry for a measurement controller 124 may include lab bench hardware such as source measure units, function generators, bias tees, chemical impedance analyzers, lock-in amplifiers, data acquisition devices, or the like, which may be coupled to a chip-based field effect biosensor 104.

The sample prep apparatus 112, in the depicted embodiment, is configured to automatically or semi-automatically prepare a sample solution 110. An assay using a chip-based field effect biosensor 104 may be used to determine a parameter relating to presence of an analyte in the sample solution, such as the presence, absence, or concentration of an analyte. Thus, preparation of the sample solution 110 may include preparing a solution in which the analyte may or may not be present. In some embodiments, a sample prep apparatus 112 may include automated dispensing equipment such as a dispensing robot and/or a fluidic system. In some embodiments, a sample prep apparatus 112 may include its own controller and user interface for setting sample prep parameters such as incubation time and temperature for the sample solution 110. In some embodiments, a sample prep apparatus 112 may be controlled via the data network 120. For example, the computing device 114 or the measurement controller 124 may control the sample prep apparatus 112.

In another embodiment, a system 100 may omit a sample prep apparatus 112, and a sample solution 110 may be manually prepared. In some embodiments, preparing a sample solution 110 may include obtaining or preparing a sample of a fluid in which an analyte may be observed (or the absence of an analyte may be detected). In some embodiments, preparing a sample solution 110 may include incubation of beads in the sample solution. In some embodiments, a sample solution 110 may be a biological sample such as blood, urine, saliva, or the like, directly obtained without further sample prep steps. In another embodiment, further sample prep steps to prepare a sample solution 110 may include the addition of reagents, concentration or dilution, heating or cooling, centrifuging, or the like. Various other or further preparation techniques may be used to prepare a sample solution 110 for use with a measurement controller 124.

The sample solution 110, in various embodiments, may include one or more types of biomolecules 108. Biomolecules 108, in various embodiments, may be any molecules that are produced by a biological organism, including large polymeric molecules such as proteins, polysaccharides, lipids, and nucleic acids (DNA and RNA) as well as small molecules such as primary metabolites, secondary metabolites, and other natural products. Biomolecules 108 or other analytes may include target moieties capable of being bound to capture moieties of beads. For example, target moieties may include biotin or a DNA sequence, and may be bound to, respectively, by a biotin-binding protein (e.g., streptavidin, avidin, neutravidin, or the like), or by an RNA guided Cas enzyme. The presence or absence of analytes bound to the beads, or related parameter may be detected when the beads are positioned within the measurement distance of a sensing surface.

The computing device 114, in the depicted embodiment, implements an analysis module 116. In various embodiments, a computing device 114 may be a laptop computer, a desktop computer, a smartphone, a handheld computing device, a tablet computing device, a virtual computer, an embedded computing device integrated into an instrument, or the like. In further embodiment, a computing device 114 may communicate with the measurement controller 124 via the data network 120. The analysis module 116, in certain embodiments, is configured to determine a parameter relating to presence of the target moiety in the sample solution 110, based on calibration and detection measurements taken by the taken by the measurement controller 124 as described below. In various embodiments, an analysis module 116 may determine various parameters relating to the presence of a target moiety, such as a such as an indication of whether or not the target moiety (or an analyte including the target moiety) is present in the sample solution, a concentration of the target moiety (or an analyte including the target moiety), or another parameter corresponding to or related to the concentration, or the like.

In the depicted embodiment, the analysis module 116 is separate from the measurement controller 124, and is implemented by a computing device 114 separate from the measurement controller 124. In another embodiment, the analysis module 116 may be partially or fully integrated with the measurement controller 124. For example, the measurement controller 124 may include special-purpose logic hardware and/or a processor executing code stored in memory to implement all or part of the analysis module 116. In some embodiments, the analysis module 116 may be implemented as an embedded processor system or other integrated circuits that form part of a chip-based field effect biosensor 104 and/or part of a chip reader device 102. In some embodiments, where an analysis module 116 is integrated with the measurement controller 124, a system 100 may omit a separate computing device 114.

The remote data repository 118, in various embodiments, may be a device or set of devices remote from the measurement controller 124 and capable of storing data. For example, the remote data repository 118 may be, or may include, a hard disk drive, a solid-state drive, a drive array, or the like. In some embodiments, the remote data repository 118 may be a data storage device within the computing device 114. In some embodiments, a remote data repository 118 may be network attached storage, a storage area network, or the like.

In some embodiments, the measurement controller 124 (e.g., a chip-based field effect biosensor 104 and/or a chip reader device 102) may include communication circuitry that transmits measurement information to the remote data repository 118. Measurement information may be measurements from chip-based field effect biosensors 104, or information about the measurements, such as calculated quantities based on the raw measurements. The analysis module 116 may communicate with the remote data repository 118 to determine one or more parameters relating to presence of a target moiety based on the information stored by the remote data repository 118. In further embodiments, the analysis module 116 may store analysis results to the remote data repository 118. In another embodiment, however, the analysis module 116 may receive measurement information from the measurement controller 124 directly or over the data network 120, and a remote data repository 118 may be omitted (e.g., in favor of local data storage).

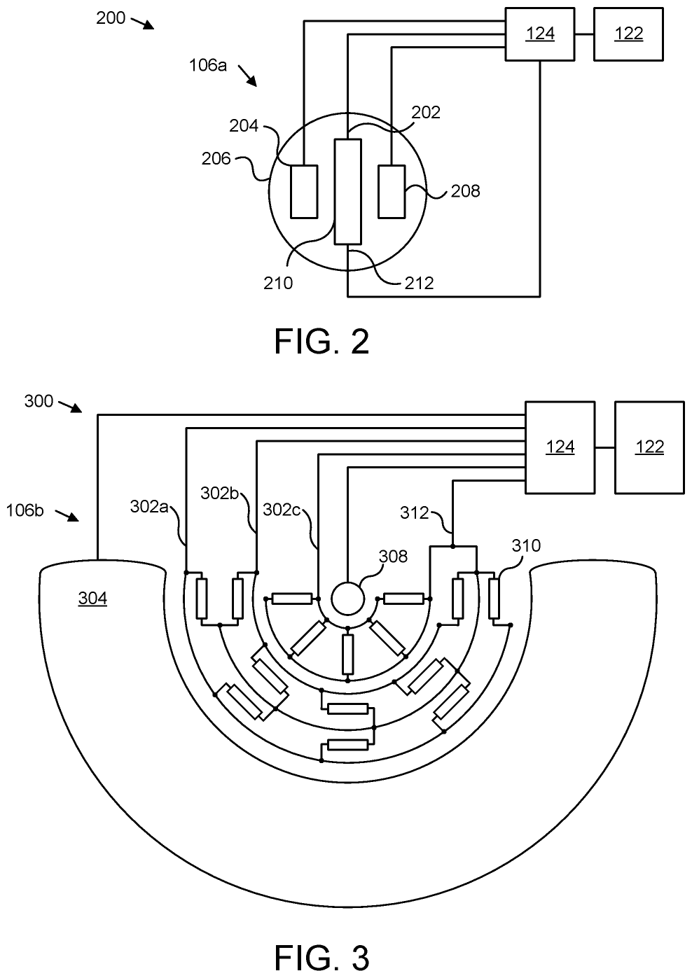

FIG. 2 is a schematic block diagram illustrating one embodiment of an apparatus 200 for transportation and detection of analytes by an enzyme, including one embodiment of a biologically gated transistor 106a, coupled to a bead control device 122 and a measurement controller 124. The biologically gated transistor 106a is depicted in a top view. The biologically gated transistor 106a, the bead control device 122, and the measurement controller 124 in the depicted embodiment may be substantially as described above with reference to FIG. 1, and are described further below.

The biologically gated transistor 106a, in the depicted embodiment, includes a source 212, a drain 202, a channel 210, a reference electrode 208, a counter electrode 204, and a liquid well 206, which are described below. In general, in various embodiments, a biologically gated transistor 106a may include at least one channel 210 capable of conducting an electrical current between the source 212 and the drain 202. As in an insulated-gate field-effect transistor, current between the source 212 and the drain 202 depends not only not only on a voltage difference between the source 212 and the drain 202 but on certain conditions that affect the conductivity of the channel 210. However, an insulated-gate field-effect transistor is a solid-state device where a gate electrode is separated from the channel by a thin dielectric layer, so that the channel conductivity is modulated by the gate-to-body (or gate-to-source) voltage. Conversely, in various embodiments, channel conductivity (and a resulting drain-to-source current) for a biologically gated transistor 106a may be modulated, gated, or affected by liquid-state events. In particular, a fluid may be applied to the biologically gated transistor 106a in contact with the channel 210, so that the channel conductivity depends on (or is gated or modulated by) a state of moieties within the fluid.

In various embodiments, the source 212, the drain 202, a channel 210, a reference electrode 208, and a counter electrode 204 may be formed on a substrate (not shown), such as an oxide or other dielectric layer of a silicon wafer or chip. Certain components of the biologically gated transistor 106a may be formed to be in contact with a fluid. For example, upper surfaces of the channel 210, the reference electrode 208 and the counter electrode 204 may be exposed or bare for direct interaction with the fluid. Other components may be covered or electrically insulated from the fluid. For example, the source 212 and drain 202 may be covered by an insulating layer such as silicon dioxide, silicon nitride, or another dielectric, so that current flows between the source 212 and drain 202 through the channel 210, without the fluid creating a short circuit or an alternative or unintended current path between the source 212 and drain 202.

The liquid well 206 may be a structure to contain the applied fluid in a region above the other components of the biologically gated transistor 106a. For example, the liquid well 206 may be a ridge of epoxy, a thermosetting resin, a thermoplastic, or the like. The liquid well 206 may be deposited on the substrate, formed as an opening in the chip packaging for the biologically gated transistor 106a, or the like.

The channel 210, in some embodiments, includes a sensing surface made of a highly sensitive conducting material such as graphene. In further embodiments, a graphene channel 210 may be deposited on the substrate for the biologically gated transistor 106a by chemical vapor deposition (CVD). In some embodiments, the channel 210 may be made from another two-dimensional material which has strong in-plane covalent bonding and weak interlayer interactions. Such materials may be referred to as van der Waals materials. For example, in various embodiments, a channel 210 may be made from graphene nanoribbons (GNR), bilayer graphene, phosphorene, stanine, graphene oxide, reduced graphene, fluorographene, molybdenum disulfide, gold, silicon, germanene, topological insulators, or the like. Various materials that conduct and exhibit field-effect properties, and are stable at room temperature when directly exposed to various solutions, may be used in a biologically gated transistor 106a. Materials that may be suitable for forming a channel 210 of a biologically gated transistor 106a may include silicon surfaces, carbon electrodes, graphene, or two-dimensional materials other than graphene. Similar materials may also be used as sensing surfaces in electrochemical or capacitive sensors. In various implementations, using a biologically gated transistor 106a with one or more channels 210 formed from planar two-dimensional van der Waals materials improves manufacturability, and lowers costs compared with one-dimensional alternatives, such as carbon nanotubes.

The source 212 and drain 202 are disposed at opposite ends of the channel 210 so that a current conducted through the channel 210 is conducted from the drain 202 to the source 212, or from the source 212 to the drain 202. In various embodiments, the source 212 and drain 202 may be made of conductive material such as gold, platinum, polysilicon, or the like. In some embodiments, the source 212 may be coupled to the substrate of the biologically gated transistor 106a (e.g., the silicon below the oxide or other dielectric layer) so that a bias voltage (or another bias signal) applied to the source 212 also biases the substrate under the channel 210. In another embodiment, a biologically gated transistor 106a may include a separate body terminal (not shown) for biasing the substrate.

The terms “source” and “drain” may be used herein to refer to conductive regions or electrodes that directly contact the channel 210, or to leads, wires or other conductors connected to those regions or electrodes. Additionally, the terms “source” and “drain” are used as the conventional names for terminals of a transistor, but without necessarily implying a type of charge carrier. For example, a graphene channel 210 may conduct electricity with electrons or holes as the charge carriers depending on various external conditions (such as the excitation conditions applied by the measurement controller 124 and the charges within the measurement distance), and the charge carriers may flow from the source 212 to the drain 202, or from the drain 202 to the source 212.

In various embodiments, one or more output signals from the biologically gated transistor 106a may be affected by excitation conditions and by charges within a measurement distance of the channel surface. As defined above, the excitation conditions may be physical, electrical, or chemical conditions applied to the biologically gated transistor 106a. Excitation conditions such as constant bias voltages (or signals), time-varying excitation voltages (or signals), temperature conditions, or the like may be applied to the biologically gated transistor 106a or to the applied fluid by the measurement controller 124. When beads incubated in the sample solution 110 are positioned within the applied fluid to be within the measurement distance of a sensing surface (e.g., the channel surface), the charges within the measurement distance may depend on whether (or to what extent) an the target moiety was captured by a capture moiety functionalized to the beads, and thus may depend on the presence, absence, or concentration of the target moiety. The interaction of such charges with the channel 210 may gate or modulate the channel conductivity, affecting one or more output signals. The output signals may be, or may include, a channel current, a voltage, a capacitance, inductance, or resistance (calculated based on applied and measured voltages and currents), a complex-valued impedance, a complex impedance spectrum, an electrochemical impedance spectrum, a Dirac voltage, a power spectral density, one or more network parameters (such as S-parameters or h-parameters), or the like.

In some embodiments, certain biomolecules or moieties may be immobilized or functionalized to the surface of the channel 210 to react with other biomolecules or moieties that may be present in the applied fluid. However, the use of beads to capture and transport analytes to be within the measurement distance may allow the analyte to be detected with a bare or unfunctionalized channel 210, or with a channel 210 that is functionalized to react to a biomolecule or moiety other than the analyte or the target moiety.

In various embodiments, a fluid applied to the channel 210 may be referred to as a liquid gate for the biologically gated transistor 106a, because one or more of the output signals for the biologically gated transistor 106a may be affected by charges within the liquid gate (e.g., charges within the measurement distance). In addition, in various embodiments, a biologically gated transistor 106a may include one or more gate electrodes for detecting and/or adjusting a voltage or electric potential of the liquid gate. For example, in the depicted embodiment, the biologically gated transistor 106a includes a reference electrode 208 for measuring an electrochemical potential of the applied fluid, and a counter electrode 204 for adjusting the electrochemical potential of the applied fluid.

In some embodiments, an electric potential may develop at the interface between the applied fluid and the reference electrode 208 and/or the counter electrode 204. Thus, in some embodiments, a reference electrode 208 may be made of a material with a known or stable electrode potential. In another embodiment, however, a reference electrode 208 may be a pseudo-reference electrode that does not maintain a constant electrode potential. Nevertheless, measurements of the electrochemical potential of the fluid via a pseudo-reference electrode may still be useful as output signals or as feedback for adjusting the electrochemical potential of the fluid via the counter electrode 204. In some embodiments, the reference electrode 208 and/or the counter electrode 204 may be made of non-reactive materials such as gold or platinum.

In some embodiments, a biologically gated transistor 106a may be made using photolithography or other commercially available chip fabrication techniques. For example, a thermal oxide layer may be grown on a silicon substrate, and metal components such as a source 212, drain 202, reference electrode 208 and/or the counter electrode 204 may be deposited or patterned on the thermal oxide layer. A graphene channel 210 may be formed using chemical vapor deposition. The use of conventional fabrication techniques may provide low-cost biologically gated transistors 106a, especially in comparison to sensors using high-cost materials such as carbon nanotubes or specialty fabrication techniques. Various other or further configurations of biologically gated transistors 106a and ways to fabricate biologically gated transistors 106a are discussed in U.S. patent application Ser. No. 15/623,279 entitled “PATTERNING GRAPHENE WITH A HARD MASK COATING”; U.S. patent application Ser. No. 15/623,295 entitled “PROVIDING A TEMPORARY PROTECTIVE LAYER ON A GRAPHENE SHEET”; U.S. patent application Ser. No. 16/522,566 entitled “SYSTEMS FOR TRANSFERRING GRAPHENE”; and U.S. Pat. No. 10,395,928 entitled “DEPOSITING A PASSIVATION LAYER ON A GRAPHENE SHEET”; each of which is incorporated herein by reference in their entireties to the extent legally allowable.

FIG. 3 is a schematic block diagram illustrating another embodiment of an apparatus 300 for transportation and detection of analytes, including another embodiment of a biologically gated transistor 106b, coupled to a bead control device 122 and a measurement controller 124. As in FIG. 2, the biologically gated transistor 106b is depicted in a top view. The biologically gated transistor 106b, the bead control device 122 and the measurement controller 124 in the depicted embodiment may be substantially as described above with reference to FIGS. 1 and 2, and are described further below.

In the depicted embodiment, the biologically gated transistor 106b includes a source 312, a plurality of drains 302, a plurality of channels 210, a reference electrode 308, and a counter electrode 304, which may be substantially similar to the source 212, drain 202, channel 210, reference electrode 208, and counter electrode 204 described above with reference to FIG. 2. (A liquid well similar to the liquid well 206 of FIG. 2 is not depicted in FIG. 3 but may similarly be provided as part of the biologically gated transistor 106b).

However, in the depicted embodiment, the biologically gated transistor 106b includes a plurality of channels 310, and a plurality of drains 302. In various embodiments, a plurality of channels 310 may be homogeneous or heterogeneous. For example, homogeneous channels 310 may be bare or unfunctionalized graphene, or may have moieties immobilized to the channels in one way. Conversely, heterogeneous channels 310 may be a mixture of bare and functionalized graphene channels 310, a mixture of channels 310 that are functionalized in more than one way (optionally including one or more unfunctionalized channels 310) or the like. For example, heterogeneous channels 310 may include a subset of unfunctionalized channels for analyte detection using beads, and another subset of channels functionalized with various moieties to perform various other or further tests. In some embodiments, providing a plurality of heterogeneous channels 310 may make a biologically gated transistor 106b useful for a variety of different tests that rely on events near the surfaces of the channels 310. Additionally, the use of multiple channels 310 may provide redundancy to mitigate damage to any individual channel 310 (e.g., mechanical damage from a pipette tip used to apply a fluid), and may make the biologically gated transistor 106b sensitive to charges in the applied fluid across a greater surface area than in a single-channel device.

In some embodiments, a biologically gated transistor 106b may include a plurality of drains 302 coupled to the channels 310. In some embodiments, one drain 302 may be provided per channel 310 so that each channel 310 can be independently biased. In some embodiments, however, channels 310 may be coupled to drains 302 in groups, so that the channels 310 of a group can be biased together in parallel, but different groups can be biased differently. For example, in the depicted embodiment, the biologically gated transistor 106b includes fifteen channels 310, coupled to three drains 302a-c, so that one of the drains 302 can be used to bias a group of five channels 310. In another embodiment, a plurality of channels 310 may be coupled in parallel to a single drain 302.

In the depicted embodiment, the channels 310 are coupled in parallel to one source 312. For some measurements, the source 312 may be coupled to ground (e.g., 0 volts, or another reference voltage). In another embodiment, however, channels 310 may be coupled to a plurality of sources 312, allowing different measurements to be made with different source biases. For example, channels 310 may be coupled to multiple sources 312 individually or in groups, as described above for the plurality of drains 302.

In the depicted embodiment, the reference electrode 308 and the counter electrode 304 are disposed so that the channels 310 are between the reference electrode 308 and the counter electrode 304. In this configuration, the electrochemical potential of the liquid gate may be modified via the counter electrode 304 and monitored via the reference electrode 308, so that the electrochemical potential near the channels 310 is close to the modified and/or monitored potential. Additionally, in the depicted embodiment, the counter electrode 304 is significantly larger than the channels 310 or the reference electrode 308, so that modifications to the electrochemical potential of the liquid gate made via the counter electrode 304 quickly occur across a large surface area, and in a large volume of the applied fluid.

Although FIGS. 2 and 3 depict individual biologically gated transistors 106a, 106b, a chip-based field effect biosensor 104 in various embodiments may include a plurality of biologically gated transistors 106 and/or capacitive sensors, which may be homogeneously or heterogeneously configured. For example, the homogeneous or heterogeneous configurations described above for multiple channels 310 in one biologically gated transistor 106b may similarly apply to multiple biologically gated transistors 106, each with their own independent source, drain, reference, and counter terminals.

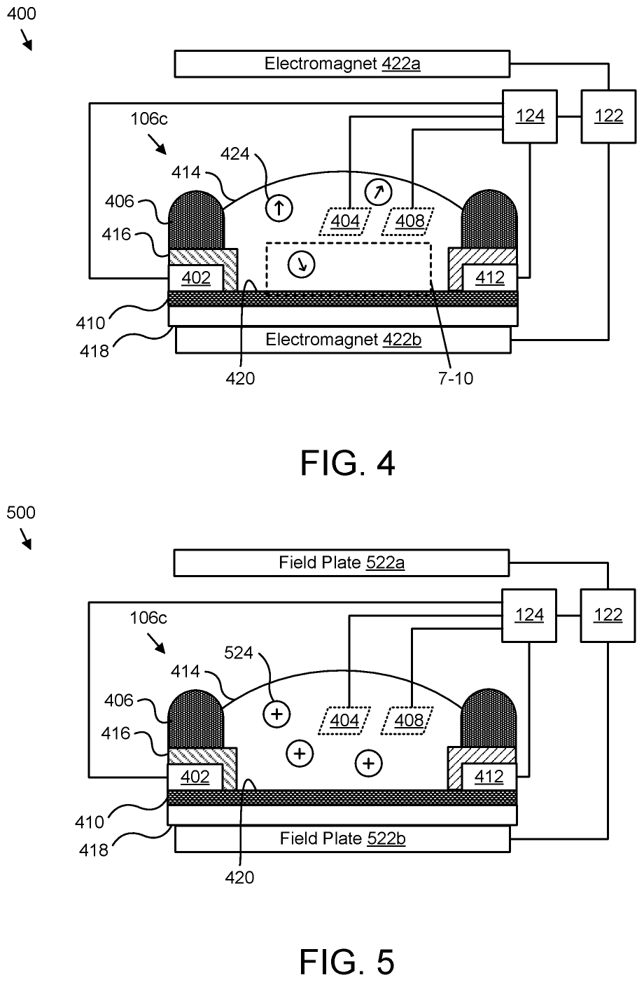

FIGS. 4 and 5 are schematic block diagrams illustrating further embodiments of apparatuses 400, 500 for transportation and detection of analytes, including embodiments of beads 424, 524 and bead control components 422, 522. In the depicted embodiments, the apparatuses 400, 500 includes a further embodiment of a biologically gated transistor 106c, coupled to a bead control device 122 and a measurement controller 124. The biologically gated transistor 106c is depicted in a cross-section view, from the side. The biologically gated transistor 106c, the measurement controller 124, the bead control device 122, the bead control components 422, and the beads 424 in the depicted embodiment may be substantially as described above with reference to FIGS. 1 through 3, and are described further below.

In the depicted embodiments, the biologically gated transistor 106c includes a source 412, a drain 402, a channel 410, a reference electrode 408, a counter electrode 404, and a liquid well 406, which may be substantially as described above. The channel 410, in the depicted embodiment, is a two-dimensional graphene region disposed on a substrate 418. The source 412 and drain 402 are formed in contact with the channel 410, and are covered by a dielectric 416 (e.g., silicon nitride). A fluid 414 is applied in contact with the surface 420 of the channel 410, which is the sensing surface 420 for a chip-based field effect biosensor 104. For example, the fluid 414 may be pipetted (or otherwise inserted) into the liquid well 406 to contact the sensing surface 420, the reference electrode 408, and the counter electrode 404. The dielectric 416 electrically insulates the source 412 and drain 402 from the fluid 414, so that current between the source 412 and drain 402 is through the channel 410 rather than directly through the applied fluid 414.

The measurement controller 124, in the depicted embodiment, is coupled to the source 412, the drain 402, the reference electrode 408, and the counter electrode 404. In various embodiments, the measurement controller 124 may apply excitation conditions to the biologically gated transistor 106c via the source 412, the drain 402, and/or the counter electrode 404. In further embodiments, the measurement controller 124 may perform measurements of one or more output signals from the biologically gated transistor 106c via the source 412, the drain 402, and/or the reference electrode 408.

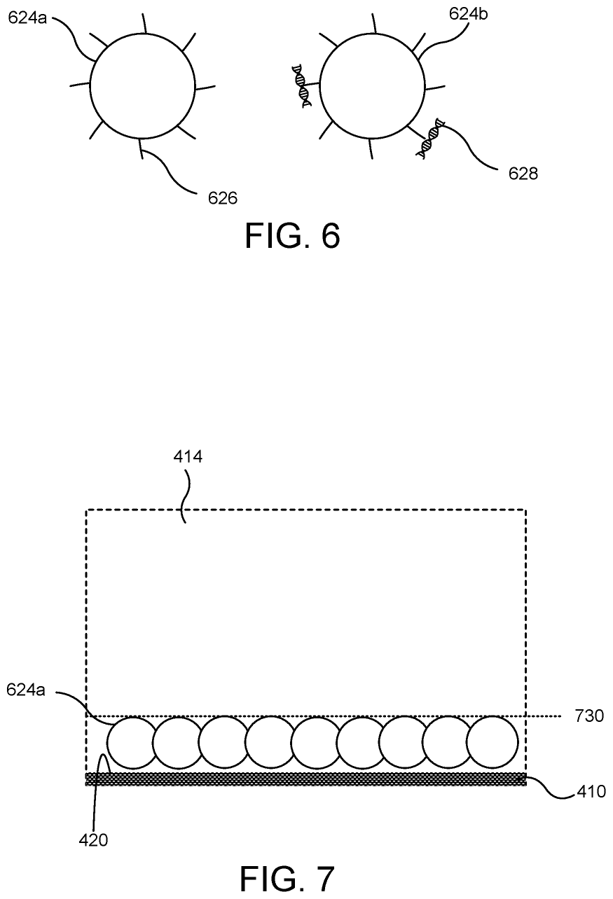

In the depicted embodiments, the fluid 414 includes a plurality of beads 424, 524 that can be electromagnetically positioned within the fluid 414 by bead control components 422, 522. Capture moieties and target moieties are not shown in FIGS. 4 and 5 so as to more clearly depict other aspects of the beads 424, 524, and bead control components 422, 522, but are described in further detail below with reference to FIG. 6.

In one embodiment, as depicted in FIG. 4, beads 424 are magnetic. Arrows on the beads 424 in FIG. 4 indicate the orientation of magnetic dipoles for the beads 424. Additionally, in the depicted embodiment, the bead control device 122 includes or is coupled to bead control components 422, which in the depicted embodiment are electromagnets 422a, 422b. As shown in FIG. 4, the bead control device 122 is not powering either of the electromagnets 422, and the beads 424 are not necessarily oriented to any particular magnetic field. For example, the magnetic interaction of the beads 424 with the earth's magnetic field may be weaker than other forces within the fluid 414. However, if the bead control device 122 turns on either electromagnet 422, the beads 424 will be oriented to the applied magnetic field, and attracted to the powered-up electromagnet 422.

With magnetic beads 424, the bead control components 422 may include a first electromagnet 422b positioned to move the beads in a first direction toward the sensing surface 420 and a second electromagnet 422a positioned to move the beads in a second direction away from the sensing surface 420. For example, in the depicted embodiment, electromagnet 422b is positioned under the sensing surface 420, and can be controlled to position beads 424 within the measurement distance of the sensing surface 420, by moving beads toward the sensing surface 420 or holding them in position. Conversely, electromagnet 422a, is positioned above the fluid 414, and can be controlled to position beads 424 further than the measurement distance of the sensing surface 420. For example, depending on the strength of the magnetic interaction between the electromagnet 422a and the beads 424 relative to the surface tension of the fluid 414, the electromagnet 422 may attract the beads 424 towards the upper surface of the fluid 414, away from the sensing surface 420, or may entirely remove the beads 424 from the fluid 414 (e.g., so that beads that have not been incubated in a sample solution can be replaced by incubated beads).

In another embodiment, as depicted in FIG. 5, beads 524 are electrically charged. A plus sign on the beads 524 in FIG. 5 indicate that the beads have a positive electric charge. However, beads in another embodiment may have a negative charge. Additionally, in the depicted embodiment, the bead control device 122 includes or is coupled to one or more bead control components 522. With charged beads 524, the bead control device 122 controls an electric field to move the beads 524. For example, in the depicted embodiment, the bead control device 122 applies an electric field using field plates 522a, 522b. The bead control device 122 may apply a voltage difference across field plates 522a and 522b so that the resulting electric field moves or positions the beads 524. Field plates 522, in various embodiments, may be any conductors to which a potential may be applied so that the potential gradient results in an electric field. For example, in the depicted embodiment, the field plates 522 are conductors above and below the biologically gated transistor 106c. In another embodiment, however, conductors within the biologically gated transistor 106c may be used to move or position electrically charged beads 524. For example, a potential applied to the channel 410 or to the substrate 418 beneath the channel may be used to attract or repel beads 524 toward or away from the surface 420. Thus, the channel 410 or substrate 418 may be used as a bead control component 522 to produce an electric field that moves beads 524.

FIG. 6 is a diagram illustrating beads 624, in one embodiment. In the depicted embodiment, beads 624 may be magnetic beads substantially similar to the magnetic beads 424 described above with reference to FIG. 4, or may be electrically charged beads substantially similar to the charged beads 524 described above with reference to FIG. 5. Beads 624, in various embodiments, may be functionalized with a capture moiety 626, to bind to a target moiety. Various capture moieties are described herein, and are represented in FIG. 6 as lines extending from the surface of the beads 624. FIG. 6 depicts two beads 624 functionalized with capture moieties 626, where a first bead 624a has not been incubated with an analyte, and where the second bead 624b has been incubated in a solution containing the analyte 628, so that one or more of the capture moieties 626 of bead 624b has bound to a target moiety of the analyte 628. In some embodiments, a target moiety may be a known moiety of an analyte 628, either because the target moiety is naturally present as a component of the analyte 628, or because the sample solution 110 has been pre-treated to bind the target moiety to the analyte 628. In the depicted embodiment, the analyte 628 is DNA, and the target moiety may be a particular sequence of nucleotides, a biotin molecule that has been linked to the DNA molecule, or the like. Various other types of analytes 628 and corresponding target moieties may be bound to by various capture moieties 626.

A capture moiety 626, in various embodiments, may be any moiety with an affinity for binding to a target moiety. Beads 624 with a particular capture moiety 626 may be selected for transport of an analyte 628 in an apparatus or system, based on a known target moiety of the analyte 628. In various embodiments, a capture moiety 626 may include antibodies, a biotin-binding protein (e.g., streptavidin, neutravidin, avidin, captavidin, or the like), biotin, zinc finger proteins or CRISPR Cas family enzymes, nucleic acids or the like. Certain capture moieties 626 may bind certain corresponding target moieties. For example, antibodies may bind to antigens, biotin-binding proteins may bind to biotin, and zinc finger proteins or CRISPR Cas family enzymes may bind to nucleic acids. Various other or further capture moieties 626 may be used to bind other or further target moieties. Capture moieties 626 may be functionalized to beads 624 by binding or linking the capture moieties to the surface of the beads 624. Various beads 624 functionalized with different capture moieties 626 may be commercially available.

FIGS. 7-10 are detail views of a region outlined in dashed lines in FIG. 4. The depicted region is above the sensing surface 420 of a chip-based field effect biosensor 104 (e.g., the surface of a channel 410 for a biologically gated transistor 106, or a working electrode surface for a capacitive electrochemical sensor). The applied fluid 414 above the sensing surface is depicted, with beads 624 as described above with reference to FIGS. 4-6 (e.g., magnetic beads or electrically charged beads). The same region is depicted at successive points in a measurement or analysis process in successive FIGS. 7-10. Capture moieties 626 depicted as lines in FIG. 6 are not depicted in FIGS. 7-10 for convenience in depicting other aspects of measurement or analysis process. Nevertheless, the beads 624 as depicted in FIGS. 7-10 are functionalized with a capture moiety 626 as described above. A dashed line indicates the measurement distance 730, so that beads 624 that are at least partially below the dashed line are within the measurement distance 730 of the sensing surface 420, and beads 624 that are fully above the dashed line are not within the measurement distance 730. In FIGS. 7-10, as in FIG. 6, the reference number 624a is used to indicate beads 624 where capture moieties are not bound to target moieties, and the reference number 624b is used to indicate beads 624 where capture moieties are bound to target moieties, so that the beads 624b are bound to analytes 628

FIG. 7 depicts a first set of beads 624, during a calibration measurement. The measurement controller 124 operates the bead control device 122 to position the beads 624 within the measurement distance 730 of the sensing surface 420. The first set of beads 624 has not been incubated in a sample solution 110, and thus the beads 624 not been exposed to or bound to the analyte 628.

In the depicted embodiment, the quantity of beads 624 in the first set of beads is sufficient to form a single layer of beads within the measurement distance 730, during the calibration measurement. In another embodiment, the quantity of beads 624 may form a partial layer of beads 624 within the measurement distance 730, leaving some of the sensing surface 420 uncovered by beads 624. In another embodiment, the quantity of beads quantity of beads 624 in the first set of beads is sufficient to form multiple layers of beads above the sensing surface 420. One or more layers may be within the measurement distance 730. For example, if the diameter of the beads 624 is approximately half of the measurement distance 730, two layers of beads 624 may stack up within the measurement distance.

To perform the calibration measurement, the measurement controller 124 uses excitation circuitry to apply excitation conditions to the biosensor 104, and uses measurement circuitry to measure one or more of the output signals from the biosensor 104 that are affected by charges within the measurement distance 730. Because the first set of beads 624 have not been incubated in the sample solution, the calibration measurement allows the measurement controller 124 to measure and record output signals that are not affected by the analyte, for later comparison to output signals that may have been affected by the analyte.

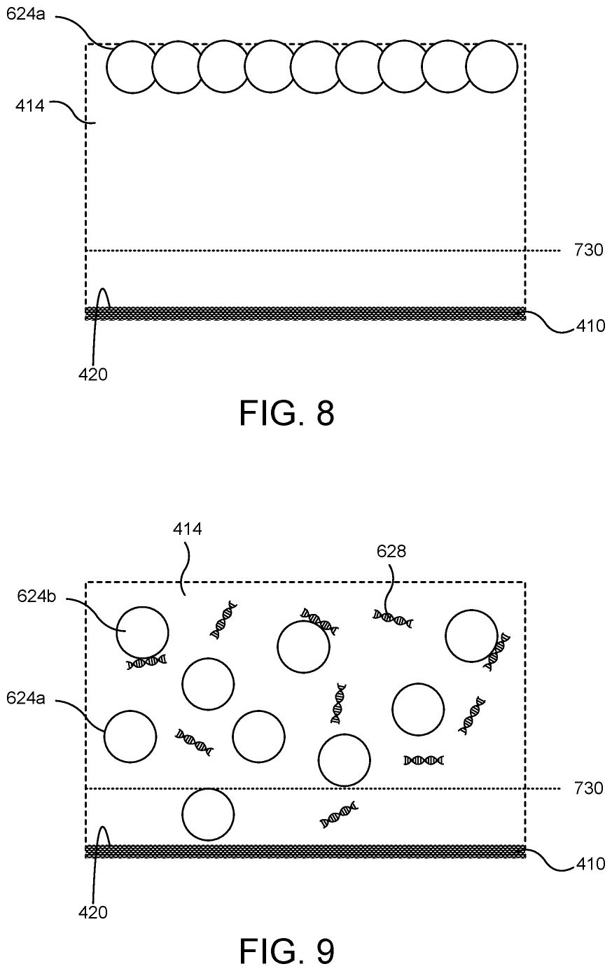

FIG. 8 depicts the first set of beads 624 removed from the sensing surface 420. The measurement controller 124 operates the bead control device 122 to move the beads 624 away from the sensing surface 420. For example, the bead control device 122 may operate an electromagnet 422a to attract magnetic beads away from the sensing surface 420, or may control an electric field to move charged beads away from the sensing surface 420. Although FIG. 8 depicts the beads 624 at the top of the depicted region to indicate that they have been removed from the sensing surface 420, actual beads 624 removed from a sensing surface 420 may be moved out of the depicted region, dispersed throughout the bulk of the fluid 414, positioned at a particular location within the fluid 414 away from the sensing surface 420, removed from the fluid 414, or the like. In various embodiments, removing the first set of beads 624 from the sensing surface 420 after the calibration measurement clears the sensing surface 420 for subsequent measurements using a second set of beads 624.

FIG. 9 depicts incubation of a second set of beads 624 in a sample solution 110. The sample solution 110 may contain an analyte 628 to be detected, or the analyte may not be present in the sample solution 110 (in which case the assay may determine that the analyte 628 is absent). Incubation of beads 624 in the sample solution allows the capture moiety 626 of the beads to bind to the target moiety of the analyte, if the analyte is in fact present in the sample solution 110.

In various embodiments, the second set of beads 624, which are incubated in the sample solution 110, may be the same set of beads as the first set of beads 624 used for the calibration measurement, or may be a different set of beads. In the depicted embodiment, the second set of beads is the same as the first set of beads. The second set in this embodiment is formed by incubating the first set of beads in the sample solution 110. For example, the first set of beads may be removed from the fluid 414 applied to the sensing surface 420 and separately incubated in the sample solution. Alternatively, as depicted in FIG. 9, the beads 624 may be incubated in situ by adding the sample solution 110 to the applied fluid 414, or by exchanging the sample solution 110 with the applied fluid 414. Bead control components 422, 522 may be used to hold beads in place during fluid exchange so that the beads 624 are not removed from the biosensor 104.

In another embodiment, the second set of beads 624 may be a different set of beads from the first set, and may be formed by incubating beads separate from the first set of beads in the sample solution 110. For example, the first set and the second set of beads 624 may respectively be different sets of non-incubated and incubated beads 624. The incubation of a separate set of beads in the sample solution 110 may take place with the sample solution 110 separate from the fluid 414 (e.g., in a separate container). The second set of beads 624 may subsequently be removed from the sample solution 110 prior to adding them to the fluid 414 applied to the sensing surface 420. In such a case, the first set of beads 624 may have been fully removed from the fluid 414 so as not to interfere with measurements involving the second set of beads 624. Incubating a second set of beads in the sample solution 110 where the second set is separate from the first set allows the incubation to take place before or during the calibration measurement (which uses the first set).

In the incubation stage, if the analyte 628 is present in the sample solution 110, the surface of the beads 624 may be exposed to the analyte, so that the capture moiety 626 of the beads 624 binds to the target moiety of the analyte 628. Thus, FIG. 9 depicts some beads 624a that have not yet bound to the analyte 628 and other beads 624b that are bound to the analyte 628. In certain embodiments, the beads in the second set of beads 624 may collectively have a greater surface area than the sensing surface 420. Additionally, as the beads move within the sample solution 110, the analyte 628 (if present) may contact the surface of the beads 624 more frequently than it contacts the sensing surface 420. Thus, functionalizing beads 624 with a capture moiety 626 instead of functionalizing the sensing surface 420 with the capture moiety 626 may provide more opportunities to bind the analyte to a surface for eventual detection. Additionally, beads 624 functionalized with a capture moiety 626 may be used with a bare or unfunctionalized sensing surface 420, allowing for multiple assays involving different capture moieties to be performed without requiring multiple types of biosensors 104.

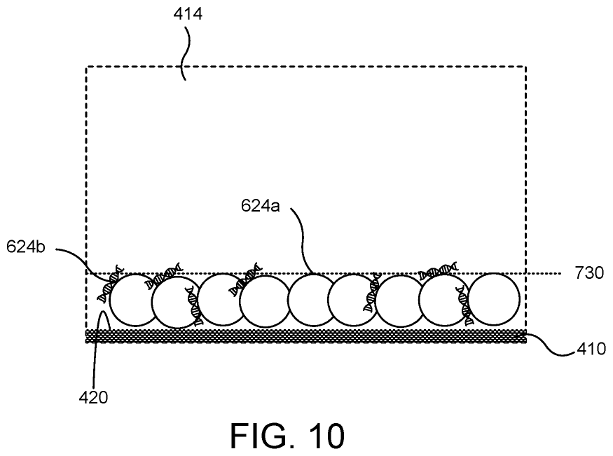

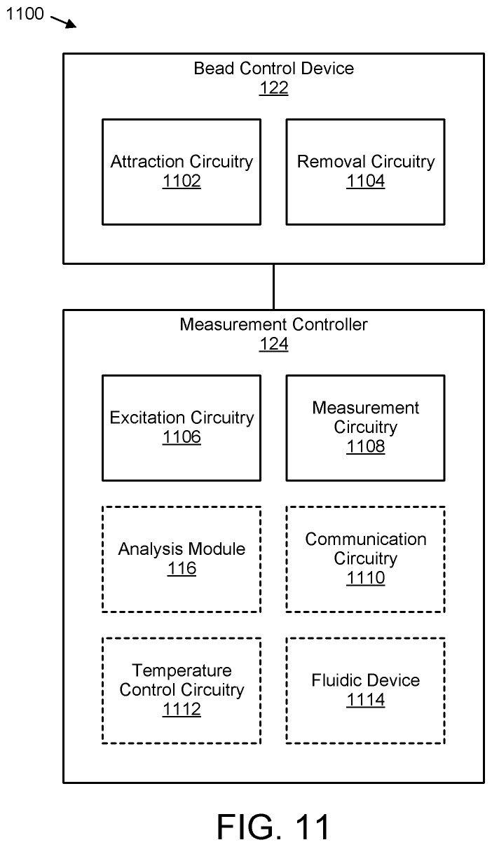

In certain embodiments, the beads 624 may be washed after incubation, and prior to performing the detection measurement described below with reference to FIG. 10. Washing the beads 624 may remove ions, molecules, or moieties that are not bound to the beads by the capture moieties 626, effectively purifying any analyte 628 bound to the beads 624, for subsequent detection. The beads may be washed in a fluid similar or identical to the fluid 414 initially applied to the biosensor for the calibration measurement. For example, the fluid 414 may be a buffer solution, purified water, or the like. Where the beads were incubated in situ by adding the sample solution 110 to the fluid, washing may include using bead control components 422, 522 may be used to hold beads in place during fluid exchange with new fluid 414. Where the beads were incubated in a separate container, washing may similarly involve magnetically or electrically securing the beads 624 so they are not washed away, while rinsing the sample solution 110 away from the beads 624.

FIG. 10 depicts the second set of beads 624 during a detection measurement. In the depicted embodiment, the analyte 628 was present in the sample solution, and is bound to at least some of the beads 624b. The measurement controller 124 operates the bead control device 122 to position the beads 624 within the measurement distance 730 of the sensing surface 420. Because the second set of beads 624 has been incubated in the sample solution 110, the analyte 628 is bound to at least some of the beads 624b. Thus, bringing the second set of beads within the measurement distance 730 also brings at least some of the analyte 628 within the measurement distance 730 of the sensing surface 420. (Conversely, if the analyte was not present in the sample solution 110, the beads will not be bound to analyte 628, and the detection measurement will be similar to the calibration measurement).

The quantity of beads 624 in the second set may be similar to the quantity in the first set, to form a single layer of beads, a partial layer of beads, or multiple layers of beads within the measurement distance, as described above with reference to the calibration measurement.

To perform the detection measurement, the measurement controller 124 uses excitation circuitry to apply excitation conditions to the biosensor 104, and uses measurement circuitry to measure one or more of the output signals from the biosensor 104 that are affected by charges within the measurement distance 730. Thus, with similar or equivalent quantities of beads 624 in the first set and the second set, and with similar or equivalent fluid 414, differences in one or more output signals between the calibration and the detection measurements may be caused by the analyte 628, if present. Greater differences between the calibration and the detection measurements may correspond to greater amounts of the analyte 628.

Thus, in certain embodiments, the analysis module 116 may determine a parameter relating to presence of the target moiety in the sample solution 110, based on the calibration measurement and the detection measurement. For example, a parameter relating to presence of the target moiety may be an indicator of the presence, absence, quantity, or concentration of the target moiety, or of the analyte containing the target moiety.

FIG. 11 is a schematic block diagram illustrating one embodiment of an apparatus 1100 for transportation and detection of analytes, including embodiments of a bead control device 122 and a measurement controller 124, which may be substantially as described above. The bead control device 122 in the depicted embodiment includes or is in communication with one or more bead control components such as electromagnets 422 or field plates 522. In the depicted embodiment, the bead control includes attraction circuitry 1102 and removal circuitry 1104.

Attraction circuitry 1102, in various embodiments, includes power circuitry and/or control circuitry (e.g., including a processor for computer control) to power and operate the bead control components to position beads 624 within a measurement distance 730 of a sensing surface 420. The attraction circuitry 1102 may be operated for calibration measurements and detection measurements to position non-incubated and incubated beads, respectively, within the measurement distance.

Removal circuitry 1104, in various embodiments, includes power circuitry and/or control circuitry (e.g., including a processor for computer control) to power and operate the bead control components to remove beads from a sensing surface 420. The removal circuitry 1104 may be operated between a calibration measurement and a detection measurement, allowing the non-incubated beads to be removed from the sensing surface 420 prior to sensing of incubated beads. The measurement controller 124 may communicate with the bead control device 122, including attraction circuitry 1102 and/or removal circuitry 1104, to position beads during and between calibration and detection measurements.

The measurement controller 124, in the depicted embodiment, includes excitation circuitry 1106 and measurement circuitry 1108. Certain components indicated by dashed lines in FIG. 11 are included in the depicted embodiment, but may be omitted in another embodiment. In the depicted embodiment, the measurement controller 124 includes an analysis module 116, communication circuitry 1110, temperature control circuitry 1112, and a fluidic device 1114. The measurement controller 124 and analysis module 116 in the depicted embodiment may be substantially as described above with reference to previous Figures.

In various embodiments, the measurement controller 124 may use excitation circuitry 1106 to apply excitation conditions to a chip-based field effect biosensor 104 that includes a sensing surface, and may use measurement circuitry 1108 to perform one or more measurements of at least one of the one or more output signals from the chip-based field effect biosensor 104. The output signal(s) may be affected by the excitation conditions, and by charges within a measurement distance of the sensing surface.

In some embodiments, the measurement controller 124 may include an analysis module 116 to determine a parameter relating to presence of a target moiety in a sample solution 110, based on the one or more measurements from the measurement circuitry 1108. In some embodiments, however, the measurement controller 124 may not include an analysis module 116. For example, in one embodiment an analysis module 116 may be implemented by a computing device 114 separate from the measurement controller 124. In some embodiments, the measurement controller 124 may include communication circuitry 1110 to transmit the measurements from the measurement circuitry 1108, or information based on the measurements, to a remote data repository 118.

The excitation circuitry 1106, in the depicted embodiment, is configured to apply one or more excitation conditions to a chip-based field effect biosensor 104, or a set of chip-based field effect biosensors 104. An excitation condition, in various embodiments, may be a physical, chemical, or electrical condition applied to biologically gated transistor 106, such as a voltage, amplitude, frequency, amplitude, phase, or waveform for an electrical or electrochemical excitation, a temperature, a fluid flow rate, or the like. Excitation circuitry 1106 may be any circuitry that applies, modifies, removes, or otherwise controls one or more excitation conditions.

In some embodiments, excitation conditions may include one or more electrical signals applied to a chip-based field effect biosensor 104 (or electrochemical potentials applied to the fluid in contact with the biosensor), such as constant-voltage biases or time-varying excitation signals. Excitation circuitry 1106 may produce biases or other excitation signals or couple them to the chip-based field effect biosensor 104 (e.g., via a source 212, drain 202, or counter electrode 204). Accordingly, in various embodiments, excitation circuitry 1106 may include any circuitry capable of generating or modulating biases or excitation signals, such as power supplies, voltage sources, current sources, oscillators, amplifiers, function generators, bias tees (e.g., to add a DC offset to an oscillating waveform), a processor executing code to control input/output pins, signal generation portions of source measure units, lock-in amplifiers, network analyzers, chemical impedance analyzers, or the like. Excitation circuitry 1106 in various other or further embodiments may include various other or further circuitry for creating and applying programmable biases.

In some embodiments, excitation conditions may include a temperature for the fluid applied to a chip-based field effect biosensor 104, and excitation circuitry 1106 may use temperature control circuitry 1112 to control the temperature. Controlling the temperature, in various embodiments, may include increasing or decreasing the temperature (e.g., to detect or analyze temperature-sensitive aspects of a biochemical interaction) maintaining a temperature in a range or near a target temperature, monitoring temperature for feedback-based control, or the like. Thus, temperature control circuitry 1112 may include any circuitry capable of changing the temperature of the fluid and/or the chip-based field effect biosensor 104. For example, in various embodiments, temperature control circuitry 1112 may include a resistive heater, a Joule heating controller to control current in a resistive heater (or in the channel 210 itself), a solid-state heat pump, a thermistor, or the like. Temperature control circuitry 1112 in various other or further embodiments may include various other or further circuitry for controlling or measuring a temperature.

Additionally, in some embodiments, excitation circuitry 1106 may include other or further circuitry for applying excitation conditions other than or in addition to electrical signals and/or temperature. For example, excitation circuitry 1106 may include electromagnets for magnetic excitation, light emitters of any desired wavelength, radioactive sources, emitters of ultraviolet light, x-rays, gamma rays, electron beams, or the like, ultrasonic transducers, mechanical agitators, or the like. Various other or further types of excitation circuitry 1106 may be used to apply various other or further excitation conditions.

As described above, one or more output signals for a chip-based field effect biosensor 104 may be affected by or sensitive to charges within the measurement distance of the sensing surface. As a simple example, with excitation conditions that include a constant drain-to-source bias voltage, charges within the measurement distance may affect an output signal, such as a drain-to-source current, a capacitance of an ionic double layer formed at the sensing surface 420 (e.g., as measured between the drain 202 and the reference electrode 208), or the like. Various output signals that may be affected by charges within the measurement distance, and measured, may include a complex resistance (e.g., impedance) of a channel 210 for a biologically gated transistor 106, electrical current through the channel 210, voltage drop across the channel 210, coupling between the channel 210 and the liquid gate (e.g., biased and/or measured via a counter electrode 204 and/or a reference electrode 208), electrical (channel) and/or electrochemical (liquid gate) voltages, currents, resistances, capacitances, inductances, complex impedances, network parameters (e.g., S-parameters or h-parameters determined using a network analyzer), a Dirac voltage (e.g., a liquid gate voltage that minimizes channel current in a graphene channel 210), charge carrier mobility, contact resistance, kinetic inductance, a spectrum based on multiple measurements such as a power spectral density, an electrical impedance spectrum, an electrochemical impedance spectrum, or the like.

Measurement circuitry 1108, in various embodiments, may include any circuitry capable of performing measurements of one or more output signals. For example, in some embodiments, measurement circuitry 1108 may include preamplifiers, amplifiers, filters, voltage followers, data acquisition (DAQ) devices or boards, sensor or transducer circuitry, signal conditioning circuitry, an analog-to-digital converter, a processor executing code to receive and process signals via input/output pins, measurement portions of source measure units, lock-in amplifiers, network analyzers, chemical impedance analyzers, or the like. Measurement circuitry 1108 in various other or further embodiments may include various other or further circuitry for performing measurements of output signals.

In various embodiments, portions or components of excitation circuitry 1106 and/or measurement circuitry 1108 may be disposed in a chip-based field effect biosensor 104, a chip reader device 102, or in a separate device (e.g., lab bench test and measurement equipment) coupled to the chip-based field effect biosensor 104. For example, single-use components such as a resistive heater component for excitation circuitry 1106 may be disposed on a chip-based field effect biosensor 104, while multi-use components such a digital signal processing circuitry for generating or analyzing complex waveforms may be disposed in a chip reader device 102. Various other ways to dispose or arrange portions or components of excitation circuitry 1106 and/or measurement circuitry 1108 may be used in various other embodiments.

The analysis module 116, in some embodiments, is configured to determine a parameter relating to presence of the target moiety, based on the calibration and detection measurements performed by the measurement circuitry 1108. Such a parameter may include an indication of whether or not the target moiety is present in the sample solution 110, a concentration of the target moiety or another parameter corresponding to or related to the concentration, or the like. In various embodiments, an analysis module 116 may use various methods, including known quantitative analysis methods to determine a parameter relating to presence of the target moiety, based on the calibration and detection measurements. Results from the analysis module 116, such as parameters characterized by the analysis module 116, may be communicated to a user directly via a display or printout (e.g., from the chip reader device 102), transmitted to a user via data network 120, saved to a storage medium (e.g., in remote data repository 118) for later access by one or more users, or the like.

In some embodiments, an analysis module 116 may be separate from the measurement controller 124. For example, an analysis module 116 may be implemented by a computing device 114 separate from the measurement controller 124. Thus, in some embodiments, a measurement controller 124 may include communication circuitry 1110, instead of or in addition to an analysis module 116. Communication circuitry 1110, in the depicted embodiment, is configured to transmit information to a remote data repository 118. The communication circuitry 1110 may transmit information via the data network 120, and may include components for data transmission (and possibly reception), such as a network interface controller (NIC) for communicating over an ethernet or Wi-Fi network, a transceiver for communicating over a mobile data network, or the like. Various other or further components for transmitting data may be included in communication circuitry 1110 in various other or further embodiments.

In some embodiments, the information transmitted by the communication circuitry 1110 to the remote data repository 118 may be information based on the measurements performed by the measurement circuitry 1108. Information based on the measurements may be the measurements themselves (e.g., raw samples), calculated information based on the measurements (e.g., spectra calculated from the raw data), and/or analysis results (e.g., a determined parameter) from the analysis module 116. In a further embodiment, an analysis module 116 may be in communication with the remote data repository 118 (e.g., via the data network 120). An analysis module 116 may be configured to characterize one or more parameters based on the information transmitted to the remote data repository 118. For example, instead of the analysis module 116 receiving measurements directly from the measurement circuitry 1108, the communication circuitry 1110 may transmit measurements (or information about the measurements) to the remote data repository 118, and the analysis module 116 may retrieve the measurements (or information about the measurements) from the remote data repository 118.

In some embodiments, storing data in a remote data repository 118 may allow information to be aggregated from multiple measurement controllers 124 for remote analysis of phenomena that may not be apparent from a single measurement controller 124. For example, for epidemiology purposes, a measurement controller 124 may determine whether a person is infected with a disease based on one or more analytes such as viruses, antibodies, DNA or RNA from a pathogen, or the like, in a sample obtained from the person, which may include a sample of blood, saliva, mucus, cerebrospinal fluid, stool, or the like. Information uploaded to a remote data repository 118 from multiple measurement controllers 124 may be used to determine aggregate characteristics, such as how infection rates differ in different geographical regions. In various embodiments, an analysis module 116 may implement various other or further ways of using aggregate information from multiple measurement controllers 124

The measurement controller 124, in various embodiments, may use excitation circuitry 1106, measurement circuitry 1108, and an analysis module 116 together in various ways with one or more chip-based field-effect biosensors 104 to determine or characterize parameters relating to presence of a target. In some embodiments, multiple chip-based field-effect biosensors 104 may be homogeneously configured (e.g., for redundancy) or heterogeneously configured (e.g., with sensing surfaces 420 functionalized in different ways to characterize different aspects of a biochemical interaction).

The fluidic device 1114, in various embodiments, may be a device used by the measurement controller 124 to drive flow of a fluid through a flow cell or other fluidic or microfluidic channels. For example, in some embodiments the measurement controller 124 may use a fluidic device 1114 to apply a fluid 414 to the sensing surface for a calibration measurement, to exchange the fluid for a sample solution for incubation of beads 624 between the calibration and detection measurements, and/or to drive flow additional fluid 414 after incubation, to remove the sample solution and wash the beads 624.

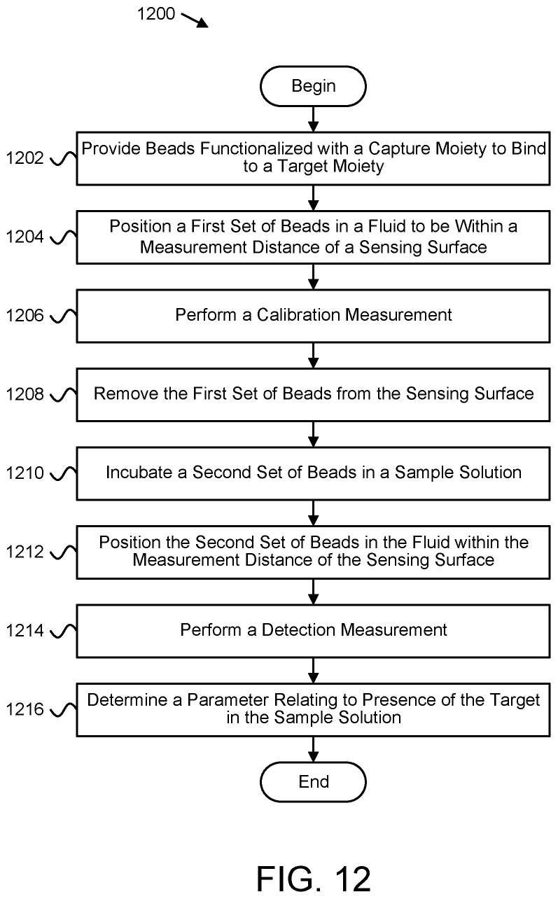

FIG. 12 is a schematic flow chart diagram illustrating one embodiment of a method 1200 or transportation and detection of analytes. The method 1200 begins with providing 1202 a plurality of beads 624 functionalized with a capture moiety 626 to bind to a target moiety. A first set of the beads 624 is positioned 1204 within a fluid 414 to be within a measurement distance 730 of a sensing surface 420 of a chip-based field effect biosensor 104. In the depicted embodiment, the first set of the beads has not been incubated in a sample solution 110. A calibration measurement is performed 1206 to measure at least one output signal from the chip-based field effect biosensor 104. The first set of beads 624 is removed 1208 from the sensing surface 420.

In some embodiments, the beads 624 may be magnetic, and positioning 1204 the first set of the beads 624 to be within the measurement distance 730 of the sensing surface 420 includes activating a first electromagnet 422b. Similarly, removing 1208 the first set of beads 624 from the sensing surface 420 may include activating a second electromagnet 422a.

In some embodiments, the beads 624 may be electrically charged, and positioning 1204 the first set of the beads 624 to be within the measurement distance 730 of the sensing surface 420 includes applying a first electric field (e.g., by applying a voltage difference across two conductors such as field plates 522). Similarly, removing 1208 the first set of beads 624 from the sensing surface 420 may include applying a second electric field (e.g., by changing the voltage of one or more conductors).

A second set of beads 624 is incubated 1210 in the sample solution 110. The second set of beads 624 is positioned 1212 within the fluid 414 to be within the measurement distance 730 of the sensing surface 420. A detection measurement is performed 1214 to measure at least one output signal. A parameter relating to presence of the target moiety in the sample solution 110 is determined 1216, based on the calibration measurement and the detection measurement, and the method 1200 ends.

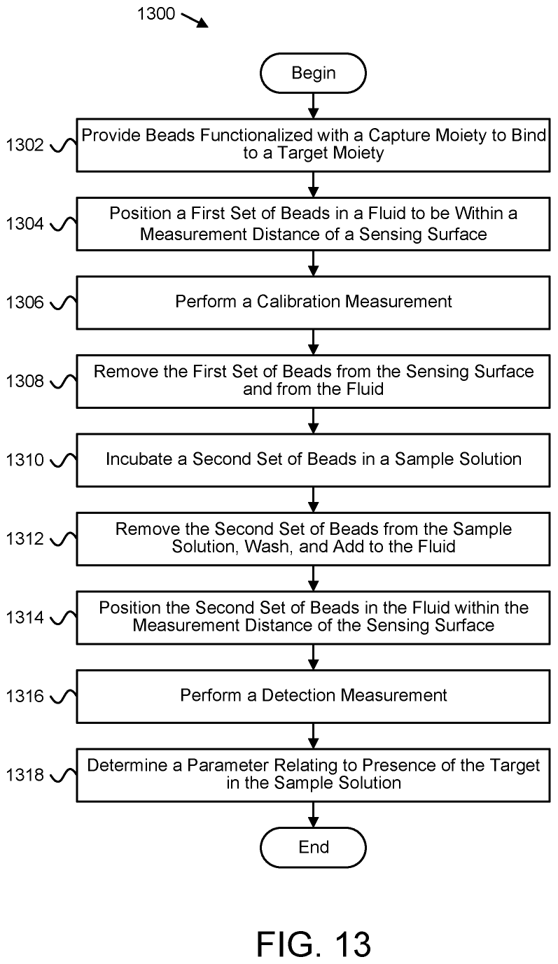

FIG. 13 is a schematic flow chart diagram illustrating another embodiment of a method 1300 for transportation and detection of analytes. Certain steps of the method 1300 may be substantially similar to steps of the method 1200 described above with reference to FIG. 12, but other steps may differ.

The method 1300 begins with providing 1302 a plurality of beads 624 functionalized with a capture moiety 626 to bind to a target moiety. A first set of the beads 624 is positioned 1304 within a fluid 414 to be within a measurement distance 730 of a sensing surface 420 of a chip-based field effect biosensor 104. In the depicted embodiment, the first set of the beads has not been incubated in a sample solution 110. A calibration measurement is performed 1306 to measure at least one output signal from the chip-based field effect biosensor 104. The first set of beads 624 is removed 1308 from the sensing surface 420, and from the fluid 414.

A second set of beads 624 is incubated 1310 in the sample solution 110. The second set of beads is removed 1312 from the sample solution, washed, and added to the fluid 414. The second set of beads 624 is positioned 1314 within the fluid 414 to be within the measurement distance 730 of the sensing surface 420. A detection measurement is performed 1316 to measure at least one output signal. A parameter relating to presence of the target moiety in the sample solution 110 is determined 1318, based on the calibration measurement and the detection measurement, and the method 1300 ends.

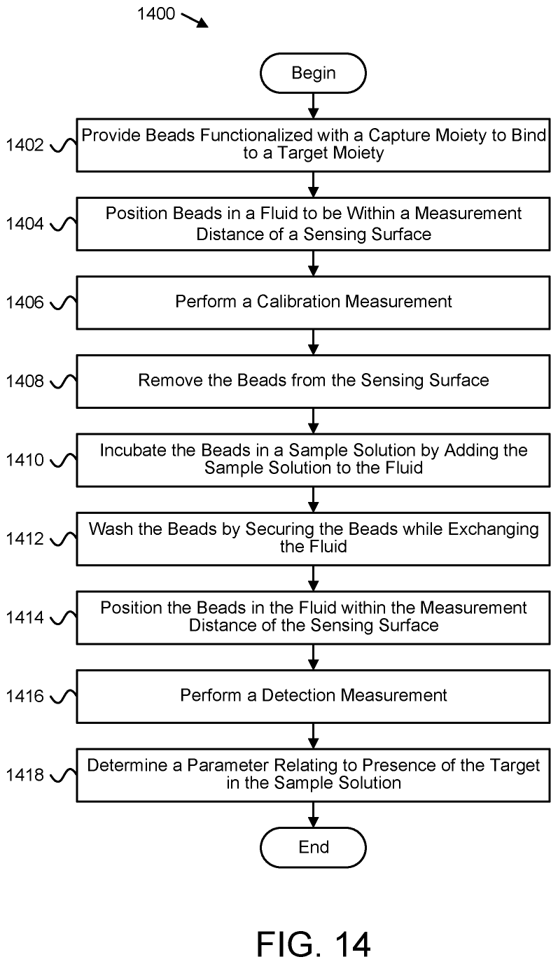

FIG. 14 is a schematic flow chart diagram illustrating another embodiment of a method 1400 for transportation and detection of analytes. Certain steps of the method 1400 may be substantially similar to steps of the method 1200 described above with reference to FIG. 12, but other steps may differ.

The method 1400 begins with providing 1402 a plurality of beads 624 functionalized with a capture moiety 626 to bind to a target moiety. A first set of the beads 624 is positioned 1404 within a fluid 414 to be within a measurement distance 730 of a sensing surface 420 of a chip-based field effect biosensor 104. In the depicted embodiment, the first set of the beads has not been incubated in a sample solution 110. A calibration measurement is performed 1406 to measure at least one output signal from the chip-based field effect biosensor 104. The first set of beads 624 is removed 1408 from the sensing surface 420, and from the fluid 414.

A second set of beads 624 is incubated 1410 in the sample solution 110, by adding the sample solution 110 to the fluid 414. The second set of beads is washed 1412 by securing the beads (e.g., using bead control components) while exchanging the fluid that has been mixed with the sample solution 110 for new fluid 414 that has not been mixed with the sample solution 110. The second set of beads 624 is positioned 1414 within the fluid 414 to be within the measurement distance 730 of the sensing surface 420. A detection measurement is performed 1416 to measure at least one output signal. A parameter relating to presence of the target moiety in the sample solution 110 is determined 1418, based on the calibration measurement and the detection measurement, and the method 1400 ends.

A means for positioning a plurality of beads 624 within a fluid 414, within a measurement distance within a measurement distance 730 of a sensing surface 430 of a chip-based field effect biosensor 104, in various embodiments, may include a bead control device 122, one or more bead control components, one or more electromagnets 422, one or more field plates or other conductors, or other means disclosed herein. Other embodiments may include similar or equivalent means for positioning beads 624.

A means for performing a calibration measurement, in various embodiments, may include a measurement controller 124, excitation circuitry 1106, measurement circuitry 1108, or other means disclosed herein. Other embodiments may include similar or equivalent means for performing a calibration measurement.

A means for performing a detection measurement, in various embodiments, may include a measurement controller 124, excitation circuitry 1106, measurement circuitry 1108, or other means disclosed herein. Other embodiments may include similar or equivalent means for performing a detection measurement.

A means for removing beads 624 from a sensing surface 420 between a calibration measurement and a detection measurement, in various embodiments, may include a bead control device 122, one or more bead control components, one or more electromagnets 422, one or more field plates or other conductors, or other means disclosed herein. Other embodiments may include similar or equivalent means for removing beads

A means for determining a parameter relating to presence of a target moiety in a sample solution 110, based on a calibration measurement and a detection measurement, in various embodiments, may include an analysis module 116, a processor executing machine-readable code with instructions for determining the parameter, other logic hardware or executable code, or other means disclosed herein. Other embodiments may include similar or equivalent means for determining a parameter.

Embodiments may be practiced in other specific forms. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A system comprising:
a chip-based field effect biosensor comprising a sensing surface, the sensing surface configured such that one or more output signals for the chip-based field effect biosensor are affected by electrical charges within a measurement distance of the sensing surface, in response to application of one or more excitation conditions to the chip-based field effect biosensor and application of a fluid in contact with the sensing surface;
a bead control device comprising one or more bead control components for electromagnetically positioning a plurality of beads within the fluid, wherein the beads are functionalized with a capture moiety to bind to a target moiety;
a measurement controller configured to operate the chip-based field effect biosensor and the bead control device to:
perform a calibration measurement of at least one of the output signals with a first set of the beads positioned within the measurement distance of the sensing surface, wherein the first set of the beads has not been incubated in a sample solution;
remove the first set of the beads from the sensing surface; and
perform a detection measurement of the at least one output signal with a second set of the beads positioned within the measurement distance of the sensing surface, wherein the second set of the beads has been incubated in the sample solution; and
an analysis module configured to determine a parameter relating to presence of the target moiety in the sample solution, based on the calibration measurement and the detection measurement.

2. The system of claim 1, wherein the beads are magnetic and the bead control components comprise a first electromagnet positioned to move the beads in a first direction toward the sensing surface and a second electromagnet positioned to move the beads in a second direction away from the sensing surface.

3. The system of claim 1, wherein the beads are electrically charged and the bead control device controls an electric field to move the beads.

4. The system of claim 1, further comprising the plurality of beads, wherein the second set of the beads is formed by incubating the first set of beads in the sample solution.

5. The system of claim 1, further comprising the plurality of beads, wherein the second set of beads is formed by incubating beads separate from the first set of beads in the sample solution.

6. The system of claim 1, wherein the chip-based field effect biosensor comprises a biologically gated transistor.

7. The system of claim 1, wherein the sensing surface comprises graphene.

8. The system of claim 1, further comprising the plurality of beads, wherein the capture moiety comprises one or more of: antibodies, a biotin-binding protein, biotin, zinc finger proteins, CRISPR Cas family enzymes, and nucleic acids.

9. A method comprising:
providing a plurality of beads functionalized with a capture moiety to bind to a target moiety;
positioning a first set of the beads within a fluid to be within a measurement distance of a sensing surface of a chip-based field effect biosensor, wherein the first set of the beads has not been incubated in a sample solution;
performing a calibration measurement of at least one output signal from the chip-based field effect biosensor;
removing the first set of the beads from the sensing surface;
incubating a second set of the beads in the sample solution;
positioning the second set of the beads within the fluid to be within the measurement distance of the sensing surface;
performing a detection measurement of the at least one output signal; and
determining a parameter relating to presence of the target moiety in the sample solution, based on the calibration measurement and the detection measurement.

10. The method of claim 9, wherein: the beads are magnetic, positioning the first set of the beads to be within the measurement distance of the sensing surface comprises activating a first electromagnet, and removing the first set of beads from the sensing surface comprises activating a second electromagnet.

11. The method of claim 9, wherein: the beads are electrically charged, positioning the first set of the beads to be within the measurement distance of the sensing surface comprises applying a first electric field, and removing the first set of beads from the sensing surface comprises applying a second electric field.

12. The method of claim 9, further comprising washing the second set of beads subsequent to incubating the second set of beads in the sample solution and prior to performing the detection measurement.

13. The method of claim 9, wherein the second set of beads is the first set of beads, and incubating the second set of the beads in the sample solution comprises adding the sample solution to the fluid.

14. The method of claim 9, wherein the second set of beads is separate from the first set of beads, and the sample solution is separate from the fluid, the method further comprising removing the second set of beads from the sample solution and adding the second set of beads to the fluid.

15. The method of claim 9, wherein the chip-based field effect biosensor comprises a biologically gated transistor.

16. The method of claim 9, wherein the sensing surface comprises graphene.

17. The method of claim 9, wherein the capture moiety comprises one or more of: antibodies, a biotin-binding protein, biotin, zinc finger proteins, CRISPR Cas family enzymes, and nucleic acids.

18. An apparatus comprising:
means for positioning a plurality of beads, within a fluid, within a measurement distance of a sensing surface of a chip-based field effect biosensor, wherein the beads are functionalized with a capture moiety to bind to a target moiety;
means for performing a calibration measurement using the chip-based field effect biosensor, with a first set of the beads positioned within the measurement distance of the sensing surface, wherein the first set of the beads has not been incubated in a sample solution; and
means for performing a detection measurement using the chip-based field effect biosensor, with a second set of the beads positioned within the measurement distance of the sensing surface, wherein the second set of the beads has been incubated in the sample solution.

19. The apparatus of claim 18, further comprising means for removing the first set of beads from the sensing surface between the calibration measurement and the detection measurement.

20. The apparatus of claim 18, further comprising means for determining a parameter relating to presence of the target moiety in the sample solution, based on the calibration measurement and the detection measurement.
